(19) United States
(12) Patent Application Publication
(10) Pub. No.: US 2005/0238733 A1
(43) Pub. Date: Oct. 27, 2005

(54) Topical anesthesia of the urinary bladder

(76) Inventor: Richard Henry, Kingston (CA)

Correspondence Address: DRINKER BIDDLE & REATH;ATTN: INTELLECTUAL PROPERTY GROUP, ONE LOGAN SQUARE, 18TH AND CHERRY STREETS, PHILADELPHIA, PA, 19103-6996, US

(21) Appl. No.: 11/149,506

(22) Filed: Jun. 10, 2005

Related U.S. Application Data
(63) Continuation of application No. 09/869,700, filed on Sep. 4, 2001, now abandoned.

(51) Int. Cl. A61K 31/24; A61K 33/00

(52) U.S. Cl. 424/717; 514/537

(57) ABSTRACT

An aqueous solution of local anesthetic is instilled into the urinary bladder in sufficient concentration with the addition of an alkalinizing agent such as sodium bicarbonate to elevate the intra-vesical pH to approximately 8.0. The combination is left in situ in the bladder for at least fifteen minutes to allow time for absorption of the base form of the local anesthetic. This method provides safe and effective topical anesthesia to allow pain-free cystoscopic biopsy and cautery of bladder lesions such as bladder cancer, and provides a means to treat inflammatory conditions of the bladder such as chronic interstitial cystitis and acute bacterial cystitis.

### BACKGROUND OF THE INVENTION

1. Field of the Invention

The invention pertains to safe and effective treatment methods for providing topical anesthetic to the urinary bladder to permit pain-free cystoscopic biopsy and cautery of bladder lesions such as bladder cancer, and to provide a means to treat inflammatory conditions of the bladder such as chronic interstitial cystitis and acute bacterial cystitis, as well as to compositions used in these treatment methods.

2. Description of the Prior Art

Topical local anesthesia of the urinary bladder remains an elusive yet desirable clinical goal for physicians diagnosing and treating a number of bladder conditions. Local anesthetics have been used in the bladder for the past forty years with limited success, restricting the physician to limited superficial resections and biopsies of small tumors. Clinical studies have confirmed the poor absorption of local anesthetics from the bladder by documenting very low blood levels of local anesthetics in patients who receive large doses of local anesthetics into their bladders. (Brian Birch and Ronald Miller, Absorption Characteristics of Lignocaine following Intravesical Instillation. Scand J of Urol Nephrol 28: 359-364, 1994, J. Brantley Thrasher, Norman Peterson and Craig Donatucci, Lidocaine as a Topical Anesthetic for Bladder Biopsies, The Journal of Urology, Vol 145, 1209-1210, June 1991, Campbell D, Adriani J. Absorption of Local Anesthetics, JAMA 1958; 168(7):873-877, Heffernan J P, Mathews R D, Sands J P, Nolan J F. Perioperative serum Bupivacaine Levels after Topical Bladder Anesthesia for Bladder Biopsy (Letter) Anesth Analg 1993; 77(2):402-403 and Pode D, Zylber-Katz E, Shapiro A: Intravesical Lidocaine: Topical Anesthesia for Bladder Mucosal Biopsies. J Urol 148:795-796,1992.)

Birch and Miller found minimal uptake of lidocaine from the bladder when 400 mg of lidocaine hydrochloride was used: plasma of levels of 0.12 micrograms/ml. They concluded that the uptake of lidocaine from the bladder is relatively poor and comparable to uptake from the urethra and intact skin, and is sixteen times less than achieved when lidocaine was administered intramuscularly, subcutaneously, and topically to the airway. They further summarized that alkalinisation of the lidocaine solution would be of doubtful value in increasing topical absorption from the bladder. (Brian Birch and Ronald Miller, Absorption Characteristics of Lignocaine following Intravesical Instillation. Scand J of Urol Nephrol 28: 359-364, 1994.)

Patients with bladder cancer are usually treated with a series of surveillance biopsies, local excisions and cautery of new lesions, which are done up to four times per year. While this surgery can be minimally invasive, it does cause significant pain and requires the physician to limit his resections or to administer systemic narcotics and sedatives to help the patient tolerate the procedure. (J. Brantley Thrasher, Norman Peterson and Craig Donatucci, Lidocaine as a Topical Anesthetic for Bladder Biopsies, The Journal of Urology, Vol 145, 1209-1210, June 1991). Excision of large tumors usually requires that the patient receive either a general anesthetic or a neuraxial block such as a spinal or epidural local anesthetic; all of which require the services of an anesthetist and an operating room and which are expensive and time consuming. With continued improvements in the equipment used for cystoscopic bladder treatments these techniques are being used more with greater success in managing bladder disease. Provision of topical anesthesia for the bladder remains one of the major problems facing physicians practicing these procedures.

Pelvic pain is a common and difficult clinical problem to evaluate and treat. Pinpointing the origin of the pain is not obvious and many diagnostic procedures and trials of treatment may be undertaken before the underlying site and nature of the problem is found. A simple and reliable means of differentiating bladder pain from other surrounding organs would be of great clinical value.

Interstitial cystitis (IC) is a chronic inflammatory disease of the urinary bladder in humans of an as yet unknown cause. Many current theories are hypothesized as to the etiology, including infection, auto-immunity, neurogenic, neuropathic and endocrine factors. (Holm-Bentzen M. Lose G: Pathology and Pathogenesis of Interstitial Cystitis. Urology 29 (Supplement):8-13 1987.) Whatever the original stimulus for this disease, mast cells in the bladder seem to be the main mechanism of ongoing disease. (Hohenfeller M, Nunues L, Schmidt R A et al: Interstitial Cystitis: Correlation with Nerve Fibres, Mast Cells and Histamine Content. Br J Urol 1993; 71:427-429.) Found in large numbers in the bladder submucosa, the mast cells are activated and appear to slowly and selectively release their secretory mediators, which result in ongoing inflammation, pain and eventually fibrosis. (Sant G R, Theoharides T C: The Role of the Mast Cell in Interstitial Cystitis. Urology Clinics of North America 21:41-53, 1994 and Theoharides T C: The Mast Cell: a Neuroendocrine Master Player. Int J Tissue React 18: 1-21, 1996)

More recently, a proliferation of nerve fibers in the bladder submucosa and detrusor muscles in IC has been demonstrated. (Christmas T L, Rode J, Chapple C R, et al :Nerve Fibre Proliferation in Interstitial Cystitis. Virchows Arch Pathol Anat 1990; 416: 447-451). The close anatomical and biochemical relationship formed between substance-P secreting neurons and mast cells, the demonstration that acetylcholine triggers bladder mast cell secretion, and the fact that pain is such a prominent feature of IC, all strongly suggest that neurohumoral triggering of mast cells may play a pivotal role in the ongoing pathogenesis of IC. (Lundeberg T, Liedberg H, Norling L, et al: Interstitial Cystitis, Correlation with Nerve Fibres, Mast Cells and Histamine. Br J Urol 71:427-429, 1993).

Local anesthetics possess a wide range of anti-inflammatory, anti-microbial and membrane stabilizing properties, in addition to their well recognized nerve conduction blocking effects. They are known to reduce neuronal transmitter release by impairing activation of presynaptic calcium channels. They may also modify post-synaptic receptors and thus inhibit the post-synaptic acetylcholine receptor. Further, the neurokinin-1 receptor, the target of the neurokinin substance-P, can be non-competitively blocked by local anesthetics.(Li Y, Wingrove D E, Too Hp et al: Local Anesthetics Inhibit Substance P Binding and Evoke Increases in Intracellular Calcium. Anesthesiology 82:166-173,1995.)

Many studies have shown that amide local anesthetics interfere with various steps of the inflammatory response of leukocytes. Lidocaine has been shown to inhibit leukocyte adherence in vitro, and to inhibit the migratory properties of these cells. (Sinclair R, Erikssson A S, Gretzer C et al: Inhibitory Effects of Amide Local Anesthetics on Stimulus-induced Human Leukocyte Metabolic Activation, LTB4 Release and IL-1 Secretion in vitro.Acta Anaes Scand 1993;37:159-165.) Lidocaine also inhibits the release of chemo-attractants, such as leukotrienes and interleukins, from activated leukocytes and consequently reduces the self-perpetuating accumulation of further leukocytes. (MacGregor R R, Thorner R E, Wright D M: Lidocaine Inhibits Granulocyte Adherence and Prevents Granulocyte Delivery to Inflammatory Sites. Blood;56:203-209,1980.) Finally, lidocaine is able to inhibit the release of histamine and other inflammatory substances from activated mast cells. However, one study has found that lidocaine at low concentrations has an opposite effect on mast cells and causes them to release their histamine content on contact with low concentrations of lidocaine (<1 mmol concentration) (Kazimierczak W, Peret M, Maslinski C: The Action of Local Anesthetics on Histamine Release. Biochemical Pharmacology 25:1747-1750, 1976) This would result in a worsening of the inflammatory condition and more pain for the patient.

There is convincing evidence that local anesthetics, at sufficient concentrations, have powerful antibacterial and anti-fungal properties. Data on growth response for E. coli bacteria treated with lidocaine and procaine firmly established that both drugs are bactericidal when concentrations of at least 2% are used. (Schmidt R M, Rosenkranz H S: Antimicrobial Activity of Local Anesthetics: Lidocaine and Procaine. The Journal of Infectious Diseases, vol 121,597-607, 1970.)

The urothelium of the bladder is almost impermeable to water, charged ions, and small molecules, such as urea and sodium (Negrete H O, Lavelle J P, Berg J, Lewis S A, & Zeidel M L: Permeability Properties of the Intact Mammalian Bladder Epithelium. Am J Physiol.271 (Renal Fluid Electrolyte Physiol.40) F886-894, 1996). This blood-urine barrier has been documented to provide a high trans-urothelial electrical resistance, which reflects the low ion flux across the urothelial membrane. (Lewis S A, Diamond J M: Active Sodium Transport by Mammalian Urinary Bladder, Nature 253:747-748,1975.) The two cellular structures responsible for this barrier function are the asymmetrical plasma membrane shielding the cytoplasm from the urine space, and the tight junctions, which close the gap between adjacent epithelial cells. Of these, the tight junctions between the cells seem to be the most important factor in protecting against absorption of urine, water, and solutes from the bladder. (Staehelin L A, Chlapowski F J & Bonneville M A: Lumenal Plasma Membrane of the Urinary Bladder. The Journal of Cell Biology: 1972:53,73-91.) This unique property of the bladder protects the animal/mammal from reabsorbing the urine that was excreted by the kidney, which would have deleterious consequences to the animal.

Local anesthetics are weak bases with a pKa range of 7.7 (etidocaine) to 9.05 (procaine). Lidocaine has a pKa of 7.9. The pKa is the pH at which 50% of the drug in an aqueous solution will be in the base form and 50% in the ion or charged form. As the pH of the solution falls, more of the drug converts into the ion form. Since the pH scale is a logarithmic scale, these changes are quite marked over a small pH range. For example, at a pH of 6.8, 5% of the lidocaine will be in the base form, while at a pH of 5.8, only 0.5% will be in the base form.

The ion and base forms of local anesthetics have important physical and physiological differences that greatly affect the pharmaco-kinetics of this class of drugs. The base or non-ionized form is highly lipid soluble and readily crosses biological membranes. It is however poorly soluble in water and will precipitate out of an aqueous solution at room temperature. Although the base form is able to cross membranes and gain access to neurons where it exerts its anesthetic effect, it is the intra-cellular ionized salt form that binds to the sodium channel in the neuron and blocks nerve conduction.

### SUMMARY OF THE INVENTION

The present invention provides a method to achieve optimum topical absorption of local anesthetics through the bladder urothelium into the submucosal region where it can exert its local anesthetic effect on the submucosal nerve plexus. The topical anesthetic formulations useful in the present invention are believed to act directly on the pH of the bladder urine to elevate it to a basic pH level; thereby optimizing absorption of the local anesthetic.

A preferred embodiment of the present invention is directed towards a single use therapy to produce deep local anesthesia of the bladder wall to allow painless cystoscopic surgical procedures such as biopsies and cautery of tumors and allow differential diagnosis of the source of pelvic pain. Another embodiment of the present invention is directed towards providing a means of treating acute bacterial infections of the bladder. Another embodiment of the present invention is directed towards chronic use of topical local anesthesia in the bladder to control the chronic neuro-inflammatory response characteristic of IC and systemic lupus erythematosis (SLE) cystitis. The anti-inflammatory effects of local anesthetics discussed above make them ideal candidates to treat the chronic inflammation of IC. The methodology of this invention allows the local anesthetic to be delivered to the desired site of action for a sufficient duration and in sufficient concentration to exert the desired anti-inflammatory effect. The antibacterial properties of local anesthetics discussed above make local anesthetics well suited to treat acute bacterial cystitis as well as the possible low-grade bacterial infection thought to be present in IC.

The ionized salt form of local anesthetics, usually a hydrochloride salt, is highly water-soluble and poorly suited to penetrate tissue and cross biological membranes. This form of the drug is especially ill suited to cross the urothelial membrane. All aqueous preparations of local anesthetics use the ionized, water-soluble form of the drug and maintain the drug in that state by lowering the pH of the solution to below pH 6 with the use of acids such as hydrochloric acid. When used to infiltrate animal tissue in order to block nerve conduction in the area, the pH of the injected solution will equilibrate with that of the surrounding tissue (usually 7.4). As the pH rises, more of the drug will assume the base form and diffuse into the surrounding tissue down the concentration gradient.

When these acidic aqueous solutions of local anesthetic are instilled into the bladder the pH of the solution equilibrates with the pH of the residual urine within the bladder. The pH of human urine varies from 4-8; but is usually in the range of 5-6. Thus, the instilled solution remains at a low pH and the local anesthetic remains in the ionized form and is unable to penetrate the bladder mucosa. This phenomenon is known as ion trapping.

According to the present invention, it has been contemplated that a means to reliably elevate the intra-vesical pH closer to the pKa of the local anesthetic (about 8), would increase the non-ionized fraction of the drug and therefore would improve bladder absorption of topical local anesthetic, making absorption more reliable and predictable. In this way, topical local anesthetic in the bladder could be used to anesthetize the bladder wall as well as provide a treatment method for a number of disorders including IC and acute bacterial cystitis.

The present method involves instilling a concentrated dose of aqueous local anesthetic into the bladder via a urinary catheter or similar device and following this with a dose of an alkalinizing buffer agent such as sodium bicarbonate. The buffer base serves two functions: it raises the pH of both the aqueous local anesthetic and the residual urine within the bladder. Sufficient buffer base is required to increase the pH to about 8.0. At a pH of around 8.0, about 50% of the local anesthetic will convert into the base form and be able to cross the bladder mucosa and diffuse down the concentration gradient into the bladder submucosal nerve plexus, which is the target site.

The present invention is based upon the discovery that there is an optimum pH in the bladder at which absorption of lidocaine is five times greater than at lower or higher pHs. Surprisingly, as the pH is increased beyond this optimum range, the absorption of lidocaine declines rapidly, providing a narrow pH range at which absorption is optimal. As the pH increases above 8.0 the lidocaine is believed to precipitate out of the aqueous solution as a higher percentage of it is converted to its lipid soluble base form before it can be absorbed into the bladder. Precipitation of the lidocaine out of solution markedly decreases the concentration of lidocaine in solution, resulting in slower absorption across the urothelial membrane.

This invention also contemplates the topical anesthesia of the bladder for diagnostic purposes. Specifically, the anesthetic is administered to a patient suffering from pelvic pain where the origin of the pain is not precisely known. This administration reliably blocks bladder pain for a temporary period of time. This allows differentiating this source of pain from pain emanating from other sources such as surrounding organs.

### BRIEF DESCRIPTION OF THE DRAWINGS

The foregoing and other objects, aspects and advantages will be better understood from the following detailed description of the preferred embodiments of the invention with reference to the drawings, in which:

FIG. 1 is a graphical depiction of the tissue levels of radioactive labeled lidocaine found in the bladder mucosa at different intra-vesical pH levels after the lidocaine was left in situ for forty five minutes in a live rabbit bladder. Tissue levels remained low at pH levels below 8.0 at around 500,000 to 1,000,000 CPM/g tissue. The peak tissue levels of around 3,500,000 CPM/g of tissue occurred at a pH between 8.1 and 8.25. At the higher pH level of 8.45, the tissue levels declined rapidly to almost the same as at the pH levels below 8.0.

FIG. 2 is a graphical depiction of the blood levels of radiolabeled lidocaine in the same anesthetized rabbits treated with the same intra-vesical radiolabeled lidocaine at different pH levels. Blood levels were found to peak at a pH of 8.1 to 800 CPM/0.1 ml of blood and remained fairly constant for the duration of the instillation time. At pH levels above and below 8.1 the absorption rate was similar in all groups and was only 25% of that at 8.1.

FIG. 3 is a graphical depiction of the blood levels of lidocaine found in human volunteers to whom intravesical lidocaine was administered with sodium bicarbonate to elevate the intra-luminal pH to 8.0. Volunteers were given either 4, 5 or 6 mg/kg 5% lidocaine with 10 cc of 8.4% sodium bicarbonate. The blood levels at regular intervals over 3 hours are measured in micrograms per milliliter of serum. All subjects attained peak levels within the desirable therapeutic range of 0.5-2 ug/cc within the first hour.

### DETAILED DESCRIPTION OF THE PREFERRED EMBODIMENTS OF THE INVENTION

Local anesthetics, all of which should be useful in the present invention, are a class of drugs which block sodium channels in nerve axons, thereby temporarily inhibiting nerve conduction. Local anesthetics are generally divided into two major subgroups based on their chemical structure. Both groups of chemicals are made up generally of an aromatic hydrophobic ring linked to a hydrophilic amino group. The linkage can be either an ester link or an amide link and it is this linkage that defines these groups. The amide group is also known as N-arylamides or carboxamides, and includes, for example, lidocaine, prilocaine, bupivacaine, ropivacaine, etidocaine, dibucaine, and mepivacaine. The ester group or aminoalkylbenzoates include, for example, cocaine, procaine, chloroprocaine, tetracaine and benzocaine.

Local anesthetics are weak bases and are highly lipid soluble in their base form. The ionic or ionized salt form is highly water soluble and best suited for stable aqueous pharmacological preparations with a low pH. The chloride or hydrochloride salt is the most commonly used salt and is the preferred preparation for this invention. Other suitable salts include bromide, sulfate, fumarate, citrate, malate, proprionate and phosphate salts.

Local anesthetics are weak bases with pKa's in the 7.7-9.1 range (e.g., pKa 7.7 for etidocaine, pKa 9.05 for procaine (Lidocaine has a pKa of 7.9)). The pKa is the pH at which 50% of the drug in solution will be in the base form and 50% in the salt form. As the pH rises above the pKa, more of the drug will dissociate and convert into the free base non-ionic form. Since the pH scale is a logarithmic scale, a rise in pH of 1.0 above the pKa would result in 90% of the drug being in the base form. Local anesthetics and their pharmacology are discussed in detail in Remington's Pharmaceutical Sciences, A. Osol, ed., Mack Pub. Co., Easton, Pa. (16^{th} ed. 1980) and the Merck Index (11^{th} ed., 1989).

Sodium bicarbonate (CHNaO₃) is a well known biological buffer that is readily soluble in water and dissociates into carbon dioxide and sodium carbonate, which in turn combine with a hydrogen ion to form water and sodium. It is this ability to take up free hydrogen ions and convert them into water that makes this buffer biologically useful. A 0.1 molar aqueous solution of sodium bicarbonate at 25 C has a pH of 8.3. Sodium bicarbonate is the preferred alkalanizing agent for use in this invention; however, other pharmaceutically acceptable buffers which can raise the pH within the bladder, and take up free hydrogen ions, may also be used in the practice of this invention.

Pharmaceutically stable solutions of local anesthetics are formulated by lowering the pH of the water to below a pH of 6. At this level, the pH of the solution is well below the pKa of the local anesthetic and almost all the drug is in the water soluble ionic salt form. When this solution is injected into animal tissue to anesthetize it, the tissue pH of 7.4 buffers the acidic fluid and the pH rises, and the local anesthetic begins to base itself into the lipid soluble form and the based form is able to penetrate the tissue, thus gaining access to the surrounding nerves where nerve conduction is temporarily blocked. When this same local anesthetic solution is instilled into the urinary bladder to provide topical anesthesia, the pH of the urine is usually in the range of 5-6. Hence, there is not the same pH rise in the urinary bladder as in other tissues which allows the local anesthetic to convert into the lipid soluble form and penetrate the tissue.

This invention provides a method to achieve bladder absorption of local anesthetic by increasing the pH in the bladder to an optimum level to maximize absorption. For exemplary purposes only, this invention utilizes a sodium bicarbonate solution having a pH between 7.5 and 8.5 to alkalinize the bladder contents. In a preferred embodiment of this invention, an injectable solution of an alkalinizing agent (e.g., sodium bicarbonate) and a local anesthetic (e.g., lidocaine) is provided in a suitable pre-filled syringe that separates these two solutions (e.g., a double barreled syringe or the like) until they are injected into the urinary bladder via a urinary catheter or other means of urethral injection. The volume of bicarbonate is preferably 5-50 milliliters (ml) and the concentration preferably ranges from 2-10%. The preferred dose is 10 ml of 8.4% sodium bicarbonate. The local anesthetic dose varies according to the specific potency of each of the specific local anesthetics and can be identified by those skilled in the art of medicine or pharmacology. In a specific example for lidocaine as the local anesthetic, the volume of lidocaine hydrochloride solution is preferably 2-20 ml and the concentration preferably ranges from 1-10%. The preferred dose is 5 ml of 10% lidocaine.

The following example demonstrates that combining an alkalinizing agent with a local anesthetic is an effective means for producing deep local anesthesia of the bladder wall to allow painless cystoscopic surgical procedures such as biopsies and cautery of tumors. This methodology can also be used for treating acute bacterial infections of the bladder. In either case, a sufficient quantity of the anesthetic is provided together with an alkalinizing agent to allow the surgical procedures to be performed or to reduce or eradicate infections. Chronic use of topical local anesthesia in the bladder is also contemplated for this invention. In this case, for example, topical anesthesia can be administered multiple times to control the chronic neuro-inflammatory response characteristic of IC and systemic lupus erythematosis (SLE) cystitis. In addition, the topical anesthesia may be administered to patients with pelvic pain to determine whether or not the pain is derived from the bladder or from other sources (e.g., surrounding organs). In this application, sufficient anesthesia is administered topically together with an alkalinizing agent. If the patient's pain subsides, it can be determined that the source of the pain is likely the bladder.

### EXAMPLE 1

Purpose: To investigate the effect of intra-vesical pH on the absorption of lidocaine from the bladder lumen into the bladder wall and blood.

Materials and Methods

Male New Zealand white rabbits (Charles River, 1.5-2 kg) were anesthetized with xylazine (5 mg/kg) and ketamine (35 mg/kg) intra-muscularly in combination with an analgesic (buprenorphine 0.03 mg/kg). Surgical plane of anesthesia was maintained over time by intra-peritoneal injection of 2 mls/kg of a 70% solution of urethane. A midline incision from the xiphoid process to the pubic symphysis was made, the ureters were ligated with 6-0 polypropylene suture and an 8 Fr pediatric feeding tube was secured in the bladder with a silk purse string around the penis. All treatments were instilled and fluids drained from the bladder using the 8 Fr catheter and light manual compression of the bladder.

Six groups of rabbits were studied. Each group was determined by the pH of the bladder rinse and the treatment fluid instilled into the bladder. Each rabbit bladder was drained and then treated with three consecutive washes of bicarbonate buffer at the appropriate pH. The six pH values investigated were: pH 6.8, 7.5, 7.78, 8.1, 8.25, and 8.46.

After the pre-treatment, 2.0×10 cpm/ml (total of 10 mls) of 14C-radio-labeled and non-radio-labeled lidocaine hydrochloride at an overall concentration of 0.4% in a pH specific bicarbonate buffer, depending on which group the rabbit was assigned to, was instilled into each bladder for 45 minutes. Samples of vena cava blood were counted using a liquid scintillation counter. After the rabbits were euthenized the bladder tissue was excised, dried, bleached and counted in a liquid scintillation counter.

Results:

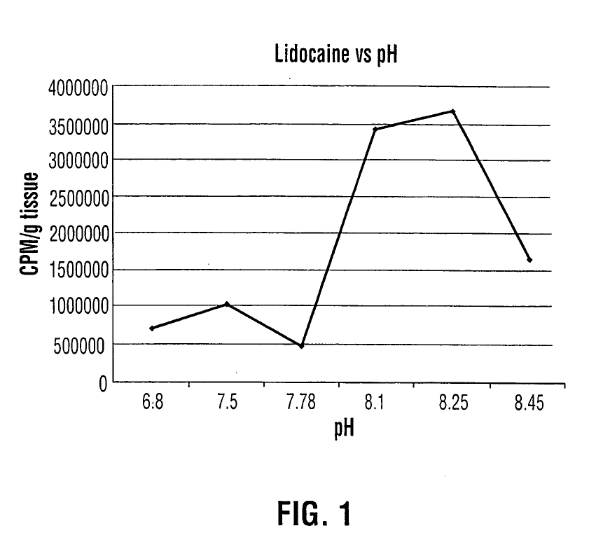

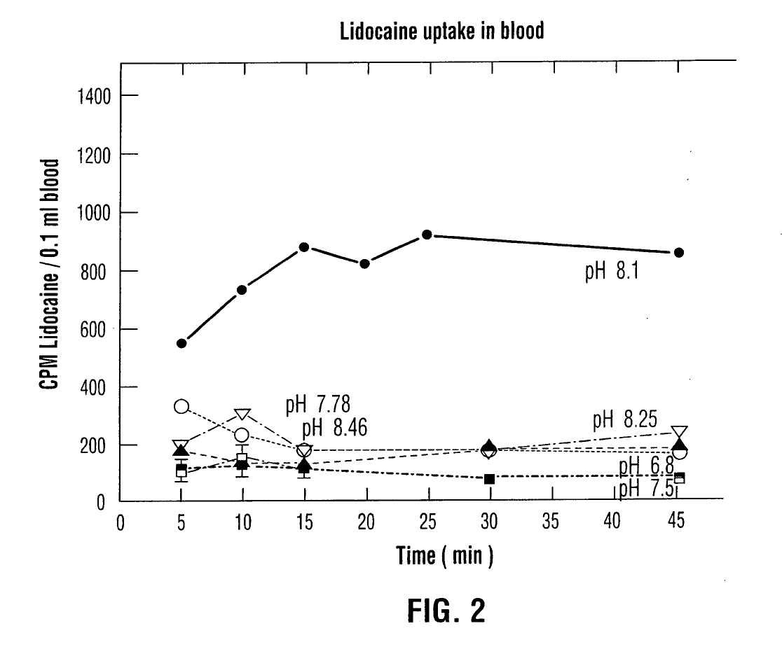

The results are presented in FIGS. 1 and 2. These Figures show the blood and tissue levels of radio-labeled lidocaine and clearly show an optimum pH level for the absorption of lidocaine (preferably between 7.8 and 8.45, and most preferably between 8.0 and 8.3). Other local anesthetics (e.g., procaine, cocaine, chloroprocaine, tetracaine, mepivacaine, lidocaine, bupivacaine, etidiocaine, ropivacaine, and benzocaine) should have similar performance characteristics with the optimum absorption varying depending on the pKa of the anesthetic used.

### EXAMPLE 2

Purpose: To determine the effect of alkalinization of intravesical lidocaine on absorption in healthy volunteers.

Methods: 12 ASA I-II healthy adult volunteers aged 18-50 years were recruited.

An in-out transurethral urinary catheter was passed up the urethra. The residual urine was drained and 5% (spinal) lidocaine was administered to three groups of four volunteers in increasing doses of 4 mg/kg, 5 mg/kg, or 6 mg/kg. This was then followed by 20 ml of 8.4% sodium bicarbonate solution after which the catheter removed. The subjects were asked to empty their bladders after one hour. Blood samples for lidocaine assay were taken at 15, 30, 60, 90, 120 and 180 minutes. The volunteers were asked to describe any side-effects felt during the three-hour observation period.

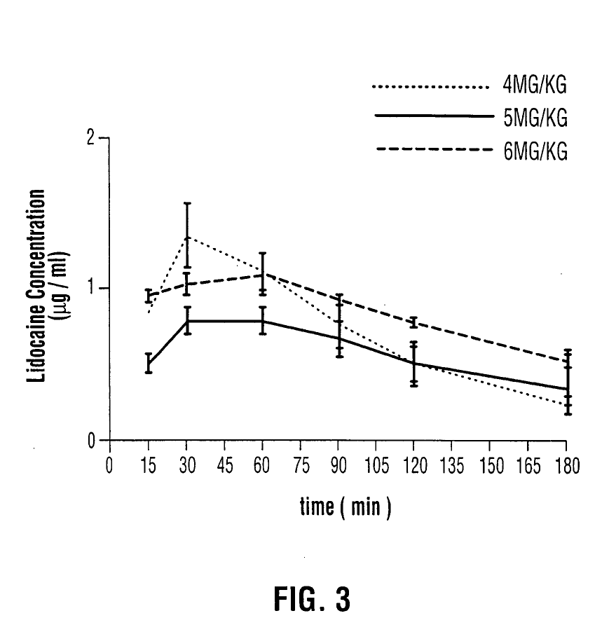

Results: In all volunteer groups, the lidocaine blood level peaked within 30 to 60 minutes of instillation. (FIG. 3) The mean peak concentration at 30 minutes was 1.06 μg/ml. The highest peak concentration in anyone patient ranged between 0.66 μg/ml and 1.71 μg/ml. After emptying the bladder the mean concentration fell to 0.40 μg/ml at 180 minutes. The pH of the voided urine at one hour was approximately 8.0 in all subjects.

Conclusion: Alkalinization of intravesical lidocaine enhances absorption as evidenced by serum lidocaine levels comparable to those obtained with tissue infiltration at similar doses. These lidocaine levels are ten times higher than previously reported and indicate an improved therapeutic effect.

1. A method for anesthetizing the bladder of a patient in need thereof, comprising the step of providing a sufficient quantity of a local anesthetic and an alkalinizing agent to the bladder of said patient to anesthetize the bladder, said local anesthetic and said alkalinizing agent being provided to said bladder separately, said local anesthetic being provided as an aqueous solution, said alkalinizing agent being provided in sufficient quantity to raise the pH of the bladder to approximately the pKA of the local anesthetic to convert at least a portion of said local anesthetic to its base form.

2. The method of claim 1 wherein said providing step is performed by instillation of said alkalinizing agent and said local anesthetic into the bladder via a catheter placed into the bladder via the urethra of said patent.

3. The method of claim 1 wherein said providing step is performed by instillation of said alkalinizing agent and said local anesthetic into the bladder percutaneously through the abdominal wall of said patient.

4. The method of claim 1 wherein said local anesthetic is selected from the group consisting of procaine, cocaine, chloroprocaine, tetracaine, mepivacaine, lidocaine, prilocaine, bupivacaine, etidiocaine, ropivacaine, and benzocaine.

5. The method of claim 1 wherein said alkalinizing agent is sodium bicarbonate.

6. The method of claim 1 wherein said local anesthetic is lidocaine and said alkalinizing agent is sodium bicarbonate.

7. The method of claim 1 wherein said alkalinizing agent provided in said providing step raises said pH of said bladder to about 8.

8. The method of claim 1 wherein said local anesthetic is provided in a sufficient concentration to reduce bacterial infectants in said bladder of said patient.

9. The method of claim 1 wherein said local anesthetic is lidocaine and is provided in a first solution in said providing step at a concentration of 1-10%, and wherein said alkalinizing agent is sodium bicarbonate and is provided in said providing step in a second solution at a concentration of 2-10% and is provided in a sufficient amount to raise said pH of said bladder to about 8, and wherein said first and second solutions being provided to said patient separately.

10. A pharmaceutical combination for anesthetizing a patient's bladder comprising:
a sufficient quantity of a local anesthetic to anesthetize said patient's bladder; and
a sufficient quantity of an alkalinizing agent to raise the pH of said patient's bladder to a level which causes the conversion of said local anesthetic to its base form, wherein said local anesthetic and said alkalinizing agent are positioned in a single-use, disposable syringe which maintains the local anesthetic and said alkalinizing agent separate until instilled in the bladder.

11. The pharmaceutical combination of claim 10 wherein said local anesthetic is selected from the group consisting of procaine, cocaine, chloroprocaine, tetracaine, mepivacaine, lidocaine, bupivacaine, etidiocaine, ropivacaine, and benzocaine.

12. The pharmaceutical combination of claim 10 wherein said alkalinizing agent is sodium bicarbonate.

13. The pharmaceutical combination of claim 10 wherein said local anesthetic is lidocaine and said alkalinizing agent is sodium bicarbonate.

14. The pharmaceutical combination of claim 10, wherein said sufficiently quantity of local anesthetic is 2 to 20 ml of 1-10% lidocaine, and wherein said sufficient quantity of alkalinizing agent is 5-50 ml of 2-20% sodium bicarbonate.

15. A method for treating interstitial cystitis, comprising the steps of periodically administering to a patient in need thereof a sufficient quantity of a local anesthetic and an alkalinizing agent, said local anesthetic and said alkalinizing agent being provided to said bladder separately, said local anesthetic and alkalinizing agent being provided to the bladder of said patient to anesthetize the bladder, said local anesthetic being provided in an aqueous solution, said alkalinizing agent being provided in sufficient quantity to raise the pH of the bladder to approximately the pKa of the local anesthetic to convert at least a portion of said local anesthetic to its base form.

16. The method of claim 15 wherein said local anesthetic is lidocaine and is provided in a first solution in said providing step at a concentration of 1-10%, and wherein said alkalinizing agent is sodium bicarbonate and is provided in said providing step in a second solution at a concentration of 2-10% and is provided in a sufficient amount to raise said pH of said bladder to about 8, and wherein said first and second solutions being provided to said patient separately.
